(19) United States
(12) Patent Application Publication
(10) Pub. No.: US 2018/0051271 A1
(43) Pub. Date: Feb. 22, 2018

(54) NON-AQUEOUS ENZYME-POLYMER CONJUGATE SOLUTIONS AND RELATED METHODS

(71) Applicant: CARNEGIE MELLON UNIVERSITY, Pittsburgh, PA (US)

(72) Inventors: Alan J. Russell, Gibsonia, PA (US); Hironobu Murata, Pittsburgh, PA (US)

(21) Appl. No.: 15/549,852

(22) PCT Filed: Feb. 10, 2016
(86) PCT No.: PCT/US16/17351

§ 371 Date: Aug. 9, 2017

Related U.S. Application Data
(60) Provisional application No. 62/176,141, filed on Feb. 10, 2015.

(51) Int. Cl. C12N 11/08; C12N 9/18; C12N 9/50; C08L 33/10; C08L 33/26

(52) U.S. Cl. CPC C12N 11/08; C12N 9/18; C08L 33/26; C08L 33/10; C12N 9/50

(57) ABSTRACT

An enzyme-polymer conjugate shows increased activity and molecular dissolution in non-aqueous solvents enabling enzyme mediated catalysis in non-aqueous solutions. The inventions described in this specification relate to enzyme-polymer conjugates, organic solutions comprising enzyme-polymer conjugates, methods of dissolving enzyme-polymer conjugates in organic solvents, and methods of using enzyme-polymer conjugates, for example, in catalysis applications. The inventions described in this specification address the problems of insolubility and decreased activity of enzymes in non-aqueous solutions comprising, for example, organic solvents or ionic liquids.

### BACKGROUND OF THE INVENTION

As nature's nanocatalysts, enzymes are able to catalyze numerous reactions with high substrate specificity in aqueous environments under mild conditions. However, enzyme activity in organic media is often several orders of magnitude below that in aqueous conditions due mainly to poor substrate binding and the structural effects of the solvent on the enzyme. In non-polar organic media, some enzymes remain structurally stable, as the solvation between nonpolar solvents and proteins favors a structured globular state. Activity in these solvents is largely reduced, however, because enzymes do not have the required mobility for catalysis. In more polar organic solvents, such as acetonitrile, water molecules are pulled away from the enzyme, which favors the denatured protein state. Even following modification, enzymes generally exist as aggregates in organic media, which allows only enzymes at the surface of the aggregate to catalyze reactions.

### SUMMARY OF THE INVENTION

The inventions described in this specification relate to enzyme-polymer conjugates, organic solutions comprising enzyme-polymer conjugates, methods of dissolving enzyme-polymer conjugates in organic solvents, and methods of using enzyme-polymer conjugates, for example, in catalysis applications. The inventions described in this specification address the problems of insolubility and decreased activity of enzymes in non-aqueous solutions comprising, for example, organic solvents or ionic liquids.

In one example, a non-aqueous solution comprises an organic solvent or an ionic liquid, wherein the organic solvent or the ionic liquid comprises no greater than 50 percent water by volume as a co-solvent, and an enzyme-polymer conjugate molecularly dissolved in the organic solvent or the ionic liquid. The enzyme-polymer conjugate comprises covalently bonded polymer chains grown from an enzyme-initiator conjugate, wherein the molecularly dissolved enzyme-polymer conjugate in the non-aqueous solution has a hydrodynamic diameter that is less than two-fold of a hydrodynamic diameter of the enzyme-polymer conjugate molecularly dissolved in water at a non-denaturing pH.

In another example, a method of catalyzing a reaction comprises dissolving an enzyme-polymer conjugate in an organic solvent or an ionic liquid to form a molecular solution of the enzyme-polymer conjugate in the organic solvent or the ionic liquid, wherein the enzyme-polymer conjugate comprises covalently bonded polymer chains grown from an enzyme-initiator conjugate, wherein the dissolved enzyme-polymer conjugate in the molecular solution has a hydrodynamic diameter that is less than two-fold of a hydrodynamic diameter of the enzyme-polymer conjugate molecularly dissolved in water at a non-denaturing pH. The method may further comprise conducting a reaction catalyzed by the enzyme-polymer conjugate in the organic solvent or the ionic liquid.

In another example, a method of catalyzing a reaction comprises providing a non-aqueous molecularly solubilized enzyme-polymer conjugate solution comprising an organic solvent or an ionic liquid to a vessel, wherein the organic solvent or the ionic liquid comprises no greater than 50 percent water by volume as a co-solvent. The molecularly solubilized enzyme-polymer conjugate comprises covalently bonded polymer chains grown from an enzyme-initiator conjugate, and the molecularly solubilized enzyme-polymer conjugate in the solution has a hydrodynamic diameter that is less than two-fold of a hydrodynamic diameter of the molecularly solubilized enzyme-polymer conjugate measured in water at a non-denaturing pH. The method may further include providing at least one reactant to the vessel and conducting a reaction catalyzed by the enzyme-polymer conjugate.

It is understood that the inventions described in this specification are not necessarily limited to the examples summarized in this Summary.

### BRIEF DESCRIPTION OF THE DRAWINGS

Various features and characteristics of the invention described in this specification may be better understood by reference to the accompanying figures, in which:

FIGS. 1A and 1B are schematic diagrams of “grafting from” synthesis of an enzyme-polymer conjugate including polymer chains that are grown from an enzyme-initiator conjugate; FIG. 1A shows the reactions producing the enzyme-initiator conjugate; FIG. 1B shows atom transfer radical polymerization (ATRP) of monomers covalently bonded to the enzyme-initiator conjugate of FIG. 1A producing an enzyme-polymer conjugate.

FIGS. 2A and 2B are schematic diagrams of “grafting from” synthesis of an enzyme-polymer conjugate comprising polymer chains covalently bonded to an esterase, wherein the polymer chains are grown from an esterase-initiator conjugate; FIG. 2A shows the reactions producing the esterase-initiator conjugate; FIG. 2B shows ATRP of monomers covalently bound to the esterase-initiator conjugate of FIG. 2A and forming the enzyme-polymer conjugate, i.e., an esterase-polymer conjugate.

FIGS. 3A and 3B are bar graphs showing the dependence of an enzyme-polymer conjugate hydrodynamic diameter (Dₕ) on solvent conditions; FIG. 3A is a graph showing the hydrodynamic diameter of an enzyme-polymer conjugate in acetonitrile with increasing water content with a fixed propanol concentration (500 mM); FIG. 3B is a graph showing the hydrodynamic diameter of the enzyme-polymer conjugate of FIG. 3A in acetonitrile with increasing propanol content with a fixed water concentration (1000 mM).

FIG. 4 is a scatter plot graph showing the effect of enzyme-polymer conjugate concentration on turbidity (absorbance at 500 nm) in acetonitrile.

FIG. 5 is a schematic diagram of the chemical reaction of enzyme-polymer conjugate catalyzed transesterification and hydrolysis of N-acetyl L-phenylalanine thiophenylester (APTE) and 1-propanol.

FIG. 6 is a schematic diagram of an enzyme-polymer conjugate as used in catalysis of the transesterification of APTE to N-acetyl L-phenylalanine propylester (APPE).

FIGS. 7A and 7B are graphs of APTE concentration versus initial rate of reaction used to determine Michaelis-Menten parameters by curve fitting; FIG. 7A is a graph of APTE concentration versus initial rate showing the dependence of the Michaelis-Menten parameters on water concentration with a fixed propanol concentration of 500 mM; FIG. 7B is a graph of APTE concentration versus initial rate showing the dependence of the Michaelis-Menten parameters on propanol concentration with a fixed water concentration of 1000 mM.

FIG. 8A is a graph of water concentration versus initial rate of reaction and the ratio of the rate of transesterification to the rate of hydrolysis in an acetonitrile solution having a fixed propanol concentration of 500 mM; and FIG. 8B is a graph of propanol concentration versus initial rate of reaction and the ratio of the rate of transesterification to the rate of hydrolysis in an acetonitrile solution having a fixed water concentration of 1000 mM.

The reader will appreciate the foregoing features and characteristics, as well as others, upon considering the following detailed description of the invention according to this specification.

### DETAILED DESCRIPTION OF THE INVENTION

The present invention provides a molecularly dissolved enzyme-polymer conjugate for use as a catalyst in organic solvents.

Various embodiments or aspects of the invention are described and illustrated in this specification to provide an overall understanding of the structure, function, operation, manufacture, and use of the disclosed compositions, systems, and methods. It is understood that the various embodiments or aspects described and illustrated in this specification are non-limiting and non-exhaustive. Thus, the invention is not limited by the description of the various non-limiting and non-exhaustive aspects or embodiments disclosed in this specification. Rather, the invention is defined solely by the claims. The features and characteristics illustrated and/or described in connection with various aspects or embodiments may be combined with the features and characteristics of other aspects or embodiments. Such modifications and variations are intended to be included within the scope of this specification. As such, the claims may be amended to recite any features or characteristics expressly or inherently described in, or otherwise expressly or inherently supported by, this specification. The various aspects or embodiments disclosed and described in this specification can comprise, consist of, or consist essentially of, or be characterized by the features and characteristics as variously described herein.

Any patent, publication, or other disclosure material identified herein is incorporated by reference into this specification in its entirety unless otherwise indicated, but only to the extent that the incorporated material does not conflict with existing definitions, statements, or other disclosure material expressly set forth in this specification. As such, and to the extent necessary, the express disclosure as set forth in this specification supersedes any conflicting material incorporated by reference herein. Any material, or portion thereof, that is said to be incorporated by reference into this specification, but which conflicts with existing definitions, statements, or other disclosure material set forth herein, is only incorporated to the extent that no conflict arises between that incorporated material and the existing disclosure material. Applicant reserves the right to amend this specification to expressly recite any subject matter, or portion thereof, incorporated by reference herein.

Reference throughout this specification to “various aspects” or “various embodiments,” or the like, refers to a particular feature or characteristic may be included in an aspect or embodiment. Thus, use of the phrase “in various aspects,” or the like, in this specification does not necessarily refer to a common aspect or embodiment, and may refer to different aspects and/or embodiments. Further, the particular features or characteristics may be combined in any suitable manner in one or more aspects or embodiments. Thus, the particular features or characteristics illustrated or described in connection with various aspects or embodiments may be combined, in whole or in part, with the features or characteristics of one or more other aspects or embodiments without limitation. Such modifications and variations are intended to be included within the scope of the present specification.

In this specification, other than where otherwise indicated, all numerical parameters are to be understood as being prefaced and modified in all instances by the term “about”, in which the numerical parameters possess the inherent variability characteristic of the underlying measurement techniques used to determine the numerical value of the parameter. At the very least, and not as an attempt to limit the application of the doctrine of equivalents to the scope of the claims, each numerical parameter described in the present description should at least be construed in light of the number of reported significant digits and by applying ordinary rounding techniques.

Also, any numerical range recited in this specification is intended to include all subranges of the same numerical precision subsumed within the recited range. For example, a range of “1.0 to 10.0” is intended to include all sub-ranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited in this specification is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein. All such ranges are intended to be inherently described in this specification such that amending to expressly recite any such sub-ranges would comply with the applicable disclosure requirements.

The grammatical articles “one”, “a”, “an”, and “the”, as used in this specification, are intended to include “at least one” or “one or more”, unless otherwise indicated. Thus, the articles are used in this specification to refer to one or more than one (i.e., to “at least one”) of the grammatical objects of the article. By way of example, “a component” refers to one or more components, and thus, possibly, more than one component is contemplated and may be employed or used in an implementation of the described embodiments. Further, the use of a singular noun includes the plural, and the use of a plural noun includes the singular, unless the context of the usage requires otherwise.

As used herein the terms “bound,” “bind”, “binding”, “associated with”, “attachment”, “attached to” and the like as used with respect to the composition, and substituents, groups, moieties, and the like of the enzyme-polymer conjugate as described herein means, unless otherwise stated, covalent or non-covalent binding, including without limitation, the attractive intermolecular forces between two or more compounds, substituents, molecules, ions or atoms that may or may not involve sharing or donating electrons. Non-covalent interactions may include ionic bonds, hydrophobic interactions, hydrogen bonds, van der Waals forces (dispersion attractions, dipole-dipole and dipole-induced dipole interactions), intercalation, entropic forces, and chemical polarity.

As used herein the term “polymer length” refers to the length of the polymer as a result of the average number of monomer residues incorporated in a polymer chain A “monomer” is a molecule that may bind chemically and covalently to other molecules to form a polymer.

As used herein the term “non-aqueous” refers to solutions comprising a solvent system comprising no greater than 50 percent water by volume.

As used herein the term “enzyme-initiator” refers to an enzyme comprising a covalently surface bonded initiator operable in Atom Transfer Radical Polymerization (ATRP), free radical polymerization, or Reversible Addition-Fragmentation chain Transfer (RAFT) polymerization.

As used herein the term “catalyst” refers to a substance that can cause a change in the rate of a chemical reaction without itself being consumed in the reaction (the changing of the reaction rate by use of a catalyst is called catalysis).

As used herein the term “enzyme” refers to any of a group of catalytic proteins that are produced by native or transgenic living cells or protein engineering, and that mediate and promote the chemical processes of life or other chemical reactions without themselves being altered or destroyed. Consonant with their role as biological catalysts, enzymes show considerable selectivity for the molecules upon which they act (called “substrates”). As used herein, the terms “active site” and “enzyme active site” refers to a specific region of an enzyme where a substrate binds and catalysis takes place (also referred to as “binding site”).

As used herein the term “inhibitor” refers to a substance that diminishes the rate of a chemical reaction often by binding within the active site of an enzyme in a process referred to as “inhibition.” In enzyme-catalyzed reactions an inhibitor frequently acts by binding to the enzyme, in which case it may be referred to as an “enzyme inhibitor.”

As used herein the term “alkyl” refers to a straight-chained or branched hydrocarbon. Examples of alkyl groups include methyl, ethyl, n-propyl, isopropyl, tert-butyl, and n-pentyl. Similarly, the term “alkenyl” or “alkynyl” refers to a straight-chained or branched hydrocarbon containing, respectively, one or more C═C double bonds or one or more C≡C triple bonds.

As used herein the terms “free enzyme,” “free lipase,” “free metalloproteinase,” “free subtilase,” or “free esterase,” or the like, refer to native-type enzymes that are not modified by polymers, radicals, combinations thereof, or the like.

As used herein the term “functional group” refers to specific groups of atoms or bonds within molecules that are responsible for the characteristic chemical reactions of those molecules. As used herein the term “phosphoryl functional group” refers to derivatives of phosphoric acid.

As used herein the term “solvent” refers to a substance that dissolves a solute resulting in a solution. A solvent can be a pure substance or a mixture. As used herein the term “solution” refers to a homogeneous mixture composed of two or more substances. As used herein the term “solute” refers to a substance dissolved in a solvent.

As used herein the term “mixture” refers to a product of a mechanical blending or mixing of chemical substances like elements and compounds, without chemical change, so that each ingredient substance retains its own chemical properties and makeup.

As used herein the term “soluble” refers to the property of a solid, liquid, or gaseous chemical solute to dissolve in a solid, liquid, or gaseous solvent to form a solution of the solute in the solvent. As used herein the terms “saturation” and “saturation concentration” refers to the extent of the solubility of the solute in the solvent, where adding more solute does not increase the concentration of the solution and begins to precipitate the excess amount of solute.

As used herein the terms “molecularly soluble,” “molecularly solubilized,” “molecular solution,” “molecularly dissolved,” and “molecularly solubilize” refer to the dissolution of enzyme-polymer conjugates into solvents such that the conjugates are separately solvated and the hydrodynamic diameter of each of the solvated conjugates as measured in the solvents is less than two-fold of the hydrodynamic diameter of the conjugates as measured in water at a non-denaturing pH. These terms exclude colloidal suspensions, emulations, or dissolved or suspended aggregates of conjugates.

As used herein the term “concentration” of a solute in a solution refers to a measure of how much of the solute is dissolved in the solvent with regard to how much solvent is present.

As used herein the term “anhydrous” refers to a substance that contains no water (e.g., proteins, enzymes, or enzyme-polymer conjugates lacking their water of hydration). As used herein the term “water of hydration” or “water of crystallization” refers to water molecules that are present on the inside of folded proteins, folded enzymes, or inside enzyme-polymer conjugates.

As used herein the term “transesterification” refers to the process of exchanging an organic group R″ of an ester with an organic group R′ of an alcohol. Transesterification reactions can be catalyzed by the addition of a catalyst in the form of an enzyme or other appropriate enzyme-polymer conjugate, an acid, or a base. Transesterification reactions may include glycerolysis, interesterification, or ester-ester interchange. “Interesterification” as used herein refers to an exchange of acyl groups between an ester and an alcohol. “Reversible transesterification reactions” refer to reactions in which an oil or fat is reacted with a monohydric alcohol in the presence of a catalyst.

As used herein an “enzyme-polymer conjugate” refers to an enzyme that has been covalently modified to graft a polymer from functional groups present on the surface of the enzyme in a catalytically active conformation. The enzyme-polymer conjugate described herein can be in the form of a salt, if applicable. A salt, for example, can be formed between an anion and a positively charged group (e.g., an amino group at a pH value below its pKₐ) on a protein-polymer conjugate of this invention. Suitable anions include chloride, bromide, iodide, sulfate, nitrate, phosphate, citrate, methanesulfonate, trifluoroacetate, and acetate. Likewise, a salt can also be formed between a cation and a negatively charged group (e.g., carboxylate) on a protein-polymer conjugate of this invention. Suitable cations include sodium ion, potassium ion, magnesium ion, calcium ion, and an ammonium cation such as tetramethylammonium ion. In addition, the enzyme-polymer conjugate may have one or more double bonds, or one or more asymmetric centers. Such a conjugate can occur as racemates, racemic mixtures, single enantiomers, individual diastereomers, diastereomeric mixtures, and cis- or trans- or E- or Z-double bond isomeric forms.

As used herein, the term “non-denaturing” pH refers to a liquid including a pH value in which the liquid favors enzymes in their catalytically active conformation.

As used herein, the term “flexibility” refers to a physical ability of a polymer, monomer, co-polymer, or the like, to bend without breaking covalent bonds in the polymer chain.

As used herein, the term “enzyme activity” refers to a measure of the quantity of active enzyme present and is equal to the number of moles of substrate converted per unit time.

As used herein, the term “specific activity” refers to a measure of the activity of an enzyme per milligram of total protein, expressed, for example, in μM/min·mg. Specific activity is also a measure of enzyme processivity, at a specific substrate concentration.

As described above, enzymes exhibit poor solubility and activity in organic solvents. Thus, there is a need for molecular dissolution of enzymes in organic solvents, which provide dramatic increases in molecularly dissolved enzyme activity as well as tight binding of substrates to enzymes in organic media

While most effective in aqueous environments, enzymes are able to catalyze reactions in essentially anhydrous organic media, although not in a molecularly dissolved state. However, enzyme activity in organic solvents is limited as a result of inefficient substrate binding, lack of solubility, and inactivation by hydrophilic anhydrous solvents. ATRP was used to synthesize an enzyme-polymer conjugate which is molecularly soluble and catalytically active in organic solvents such as, for example, acetonitrile.

Enzyme-polymer conjugates, for example, are useful for a variety of applications, due to increased activity and solubility in organic solvents. The activity and stability response of polymer-based protein-engineered enzymes to a variety of stressors (pH, temperature, protease degradation) in aqueous media was examined specifically, enzyme-polymer conjugates prepared by ATRP, are molecularly soluble in organic solvents such as, for example, acetonitrile.

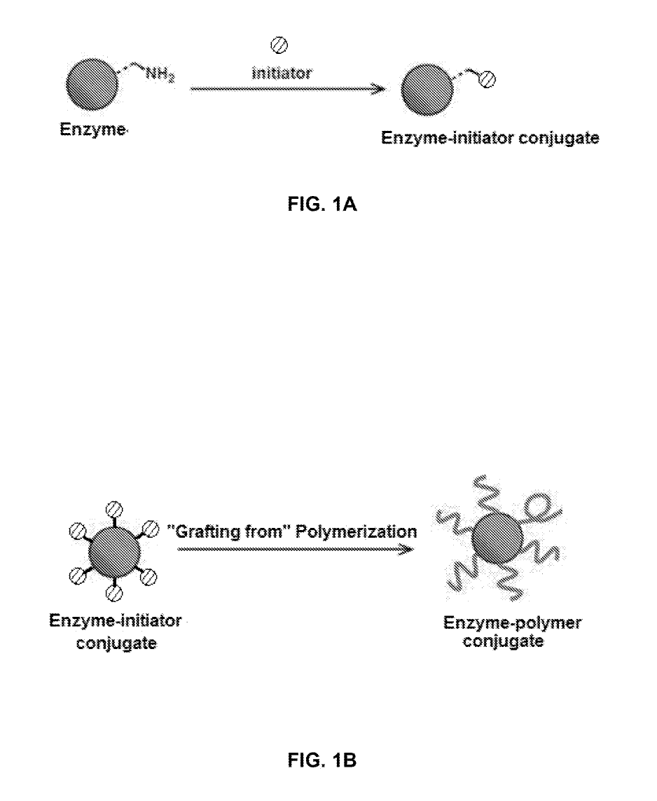

FIGS. 1A and 1B show schematics of the synthesis of an enzyme-polymer conjugate synthesized using the polymer-based protein engineering (PBPE) polymerization method, ATRP, and a “grafting-from” or “grown from” approach. To provide an alternative approach to the synthesis of enzyme-polymer conjugates that allows for higher grafted chain densities, finer grafting site control, and predictability, the protein surface-initiated “grafting-from” technique was developed where an initiator was first covalently bound to the enzyme (see FIG. 1A) followed by polymer synthesis using the protein as an enzyme-initiator conjugate (see FIG. 1B) to produce the enzyme-polymer conjugate.

In the “grafting-from” or “grown from” approach a wide range of monomers can be used to form the polymer covalently bonded to the enzyme, and the molecular weight of the grafted polymer on the enzyme can be controlled to a narrow molecular weight distribution. Thus, conjugates produced using a “grafting-from” or “grown from” approach can potentially be rationally designed, allowing structure-function relationships between the polymer and the enzyme to predict the functionality of the covalently bonded enzyme-polymer conjugate.

In various aspects, the polymer component of an enzyme-polymer conjugate, for example, poly(2-(dimethylamino)ethyl methacrylate) (pDMAEMA), may be used to evaluate how “grafted-from” stimuli-responsive enzyme-polymer conjugates can be controlled with environmental variables such as solvent composition. Polymers other than pDMAEMA may also be used, including polymers produced from radically-polymerizable monomers, examples of which include, but are not limited to, poly(oligo(ethylene glycol) methyl ether methacrylate) (pOEGMA), poly(dimethylacrylamide) (pDMAA), or liquid polymer, or combinations of any thereof.

The present invention advances the understanding of enzymatic mechanisms as well as enzyme-polymer interactions, especially those involved in catalysis in organic solvents. To the inventors' knowledge, the molecular dissolution of any enzyme in acetonitrile has not been described previously. Further, to date, the molecular dissolution of esterase enzymes in an organic solvent or ionic liquid has not been previously described.

In various aspects, the present invention describes a non-aqueous solution comprising the organic solvent or ionic liquid, wherein the organic solvent or the ionic liquid comprises no greater than 50 percent water by volume as a co-solvent, and an enzyme-polymer conjugate molecularly dissolved in the organic solvent or ionic liquid. The enzyme-polymer conjugate comprises covalently bonded polymer chains grown from an enzyme-initiator conjugate. The molecularly dissolved enzyme-polymer conjugate in the non-aqueous solution has a hydrodynamic diameter that is less than two-fold of a hydrodynamic diameter of the enzyme-polymer conjugate molecularly dissolved in water at a non-denaturing pH.

In various aspects, the organic solvent used to molecularly dissolve the enzyme-polymer conjugate may include an anhydrous organic solvent or an ionic liquid. In some aspects, the organic solvent may include water as a co-solvent up to 50% by volume, up to 45% by volume, up to 40% by volume, up to 35% by volume, up to 30% by volume, up to 25% by volume, up to 20% by volume, up to 15% by volume, up to 10% by volume, up to 5% by volume, up to 1% by volume, or up to 0.5%. In various aspects, the organic solvent or ionic liquid may include polar or hydrophilic solvents, such as, for example, acetonitrile, chloroform, tetrahydrofuran, 1,4-dioxane, an ether, hexane, toluene, or acetone, or combinations of any thereof.

The engineered structure of the enzyme-polymer conjugate may include covalently bonded polymer chains grown from an enzyme-initiator conjugate. The enzyme-polymer conjugates capable of molecular solubility in organic solvents or ionic liquids may comprise a polymer or polymers covalently bonded to an enzyme, such as, for example, an enzyme from the esterase group comprising a metalloproteinase, a subtilase, or a lipase, or combinations of any thereof. Although CT is one esterase used herein as an example of the invention, other protein enzymes may be used including, but not limited to, lipase, triacylglycerol lipase, subtilase, metalloproteinase, cholinesterase, acetylcholinesterase, butyrylcholinesterase, trypsin, subtilisin, thermolysin, or CT, or combinations of any thereof. Alternative names for acetylcholinesterase are known to persons having ordinary skill in the art. For example, alternative names for acetylcholinesterase include RBC cholinesterase, erythrocyte cholinesterase, serum cholinesterase, acetylcholine acetylhydrolase, acetylhydrolase, and forms of acetylcholinesterase encoded by the AChE gene(s), AChE, AChET, AChEH, and AChER. Alternative names for butyrylcholinesterase are also known to persons having ordinary skill in the art. For example, alternative names for butyrylcholinesterase include BChE, BuChE, pseudocholinesterase, plasma cholinesterase, or acylcholine acylhydrolase. Examples of enzyme-polymer conjugates are further described in the International Publication Number WO 2015/051326 A1, the contents of which is incorporated by reference into this specification.

In various aspects, the enzyme-polymer conjugate composition may comprise at least one polymer exhibiting a polymer length ranging from a minimum of at least 2 monomer repeats to about 1000 monomer repeats. For example, the polymer length may range from a minimum of at least 5 monomer repeats to about 750 monomer repeats, from a minimum of at least 25 monomer repeats to about 500 monomer repeats, from a minimum of at least 100 monomer repeats to about 250 monomer repeats, or any range subsumed therein, for example, from a minimum of 10 monomer repeats to about 900 monomer repeats.

In various aspects, the enzyme-polymer conjugate composition may comprise a co-polymer comprising more than one monomeric repeating unit. In various aspects, the enzyme-polymer conjugate may comprise at least one polymer that is a co-polymer comprising at least two different monomers, wherein at least one monomer comprises a member selected from the group consisting of aldoximes, ketoximes, muco-adhesion monomers, polyethylene glycol, bis-pyridinium oximes, N,N-dimethylacrylamide, N-isopropylacrylamide, (meth)acrylate, N,N-dimethylaminoethyl methacrylate, carboxyl acrylamide, 2-hydroxylethylmethacrylate, N-(2-hydroxypropyl)methacrylamide, quaternary ammonium monomers, sulfobetain methacrylate, oligo(ethylene glycol) methyl ether methacrylate, 2-PAM monomers, 4-PAM monomers, Clickable azide monomers, and the like, and combinations thereof.

In addition, the co-polymer of the enzyme-polymer conjugate may comprise at least two different monomers, wherein at least one monomer may comprise a varied topology from at least one different monomer of the co-polymer. More specifically, the varied topology of the at least one monomer may include block, random, star, end-functional, or in-chain functional co-polymer topology. For example, at least one monomer of the co-polymer may include at least one monomer of a di-block topology. The co-polymers, monomers for di-block formation, monomers including an end functional group, or in-chain functional co-polymers may be synthesized utilizing the materials and methods described in U.S. Pat. No. 5,789,487 to Matyjaszewski et al, U.S. Pat. No. 6,624,263 to Matyjaszewski et al, U.S. Patent Application Publication No. 2009/0171024 to Jakubowski et al., and Matyjaszewski, K, and Davis, T. P., ed., Handbook of Radical Polymerization, John Wiley and Sons, Inc., Hoboken, N.J. (2002), each of which is incorporated by reference into this specification.

In various aspects, the enzyme-polymer conjugate may include a plurality of polymers each covalently bonded and thus conjugated to the enzyme, each polymer comprising a plurality of monomer units wherein at least one said monomer unit comprises an oxime functional group and wherein a plurality of monomer units of each polymer comprises an oxime functional group. In various aspects, a plurality of monomer units of each polymer of the plurality of polymers may comprise an oxime functional group. In various aspects, the plurality of polymers may comprise co-polymers wherein each co-polymer may include at least two different monomers in which at least one monomer comprises a member selected from the group consisting of aldoximes, ketoximes, muco-adhesion monomers, polyethylene glycol, bis-pyridinium oximes, N,N-dimethylacrylamide, N-isopropylacrylamide, (meth)acrylate, N,N-dimethylaminoethyl methacrylate, carboxyl acrylamide, 2-hydroxylethylmethacrylate, N-(2-hydroxypropyl)methacrylamide, quaternary ammonium monomers, sulfobetain methacrylate, oligo(ethylene glycol) methyl ether methacrylate, 2-PAM monomers, 4-PAM monomers, Clickable azide monomers, and the like, and combinations thereof.

Where the enzyme-polymer conjugate comprises a co-polymer, the co-polymer may comprise a member of the group consisting of a statistical co-polymer, a random co-polymer, an alternating co-polymer, a block co-polymer, a di-block co-polymer, a tri-block co-polymer, a graft co-polymer, a multiple-block co-polymer, or the like, or combinations thereof.

In various aspects, the enzyme-polymer conjugate may comprise a plurality of polymers each covalently conjugated to the enzyme and each polymer may comprise a plurality of monomer units wherein at least one said monomer unit comprises an oxime functional group and the plurality of polymers comprises a plurality of co-polymers and a plurality of homopolymers. Further, each co-polymer of the plurality of co-polymers may comprise at least two different monomers, wherein at least one monomer comprises a member selected from the group consisting of aldoximes, ketoximes, muco-adhesion monomers, polyethylene glycol, bis-pyridinium oximes, N,N-dimethylacrylamide, N-isopropylacrylamide, (meth)acrylate, N,N-dimethylaminoethyl methacrylate, carboxyl acrylamide, 2-hydroxylethylmethacrylate, N-(2-hydroxypropyl)methacrylamide, quaternary ammonium monomers, sulfobetain methacrylate, oligo(ethylene glycol) methyl ether methacrylate, 2-PAM monomers, 4-PAM monomers, Clickable azide monomers, and the like, and combinations thereof. In addition, each homopolymer of the plurality of homopolymers comprises a member selected from the group consisting of aldoximes, ketoximes, muco-adhesion monomers, polyethylene glycol, bis-pyridinium oximes, N,N-dimethylacrylamide, N-isopropylacrylamide, (meth)acrylate, N,N-dimethylaminoethyl methacrylate, carboxyl acrylamide, 2-hydroxylethylmethacrylate, N-(2-hydroxypropyl)methacrylamide, quaternary ammonium monomers, sulfobetain methacrylate, oligo(ethylene glycol) methyl ether methacrylate, 2-PAM monomers, 4-PAM monomers, Clickable azide monomers, and the like, and combinations thereof.

In various aspects, the molecularly dissolved enzyme-polymer conjugate in the non-aqueous solution has a hydrodynamic diameter that is less than two-fold of a hydrodynamic diameter of the enzyme-polymer conjugate molecularly dissolved in water at a non-denaturing pH. For example, in one aspect, the molecularly dissolved enzyme-polymer conjugate, esterase-pDMAEMA, in acetonitrile has a hydrodynamic diameter that is less than two-fold of the hydrodynamic diameter of the enzyme-polymer conjugate, esterase-pDMAEMA, as measured in water above pH 8 and below the low critical solution temperature.

In various aspects, the molecularly dissolved enzyme-polymer conjugate comprises an enzyme-polymer conjugate capable of catalyzing a reaction exhibiting a k_{cat} of at least 3.7 min⁻¹. In some aspects, the molecularly dissolved enzyme-polymer conjugate comprises an enzyme-polymer conjugate capable of catalyzing a reaction exhibiting a k_{cat} of at least 4 min⁻¹, of at least 5 min⁻¹, of at least 6 min⁻¹, of at least 7 min⁻¹, of at least 8 min⁻¹, of at least 9 min⁻¹, of at least 10 min⁻¹, of at least 15 min⁻¹, of at least 16 min⁻¹, of at least 18 min⁻¹, of at least 20 min⁻¹, of at least 21 min⁻¹, or of at least 25 min⁻¹.

In various aspects, the present invention includes a method of catalyzing a reaction comprising dissolving an enzyme-polymer conjugate in an organic solvent or an ionic liquid to form a molecular solution of the enzyme-polymer conjugate in the organic solvent or the ionic liquid, wherein the enzyme-polymer conjugate comprises covalently bonded polymer chains grown from an enzyme-initiator conjugate. The dissolved enzyme-polymer conjugate in the molecular solution has a hydrodynamic diameter that is less than two-fold of a hydrodynamic diameter of the enzyme-polymer conjugate molecularly dissolved in water at a non-denaturing pH. The method described further includes conducting a reaction catalyzed by the enzyme-polymer conjugate in the organic solvent or the ionic liquid.

In various aspects, an esterase is used in the synthesis of the enzyme-polymer conjugate, however, other enzymes may be used including but not limited to cholinesterase, acetylcholinesterase, butyrylcholinesterase, subtilase, subtilisin, thermolysin, lipase, triacylglycerol lipase, metalloproteinase, chymotrypsin, α-chymotrypsin, or trypsin, or combinations of any thereof. In various aspects, the enzyme-polymer conjugate may comprise an esterase-polymer conjugate comprising a chymotrypsin-pDMAEMA (CT-pDMAEMA) conjugate, a metalloproteinase-pOEGMA conjugate, a thermolysin-pOEGMA conjugate, a subtilisin-ionic liquid polymer conjugate, a subtilase-ionic liquid polymer conjugate, or a lipase-pDMAA conjugate, or combinations of any thereof.

In various aspects, the enzyme-polymer conjugate is molecularly dissolved in the organic solvent or ionic liquid. For example, the enzyme-polymer conjugate may be dissolved in an organic solvent or ionic liquid that may comprise an anhydrous organic solvent, a polar or hydrophilic solvent, or a solvent forming a non-aqueous solution, comprising, for example, acetonitrile, chloroform, tetrahydrofuran, 1,4-dioxane, an ether, hexane, toluene, or acetone, or combinations of any thereof.

In various aspects, the molecular solution used to catalyze a chemical reaction may include water up to 50% by volume. In some aspects, the molecular solution used to catalyze a chemical reaction may include water up 2000 mM. For example, in some aspects, the molecular solution may include water in an amount of 500 mM, 750, mM, 1000 mM, 1250 mM, 1500 mM, 2000 mM, or greater than 2000 mM, up to a maximum of 50% by volume.

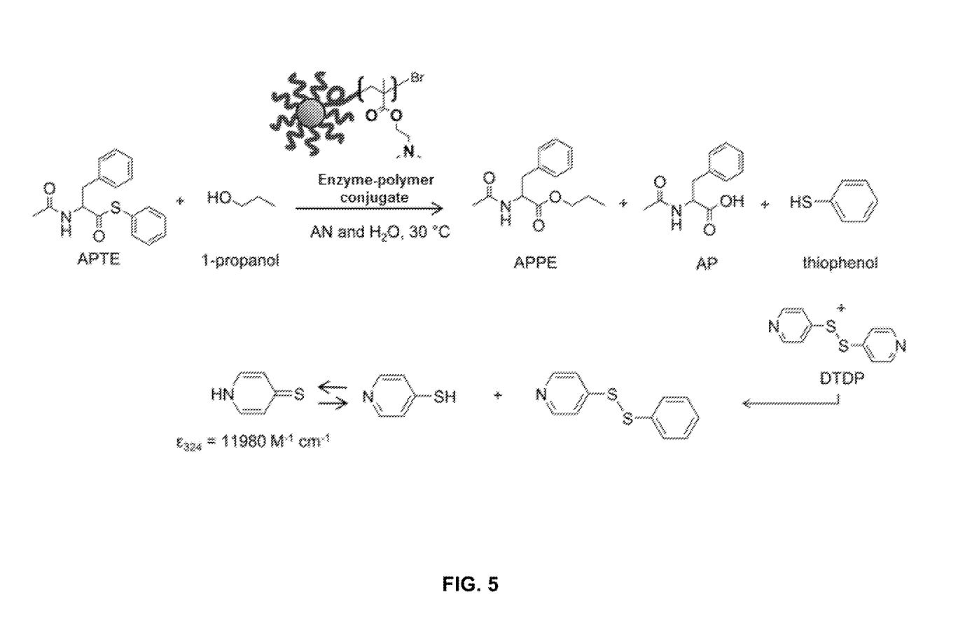

In various aspects, the chemical reaction catalyzed by the enzyme-polymer conjugate may include, for example, a transesterification reaction, a hydrolysis reaction, an enantioselective reaction, a redox reaction, a condensation reaction, a polyester synthesis reaction, or a peptide synthesis reaction, or combinations of any thereof. For example, in one aspect, an esterase-polymer conjugate such as CT-pDMAEMA can be used to catalyze the transesterification of N-acetyl L-phenylalanine thiophenylester (APTE) with 1-propanol in acetonitrile (AN), see FIG. 5.

In various aspects, the chemical reaction catalyzed by the enzyme-polymer conjugate exhibits a k_{cat} of at least 3.7 min⁻¹. In some aspects the chemical reaction catalyzed by the enzyme-polymer conjugate exhibits a k_{cat} of at least 4 min⁻¹, of at least 5 min⁻¹, of at least 6 min⁻¹, of at least 7 min⁻¹, of at least 8 min⁻¹, of at least 9 min⁻¹, of at least 10 min⁻¹, of at least 15 min⁻¹, of at least 16 min⁻¹, of at least 18 min⁻¹, of at least 20 min⁻¹, of at least 21 min⁻¹, or of at least 25 min⁻¹.

In various aspects, the present invention includes a method of catalyzing a reaction comprising providing a non-aqueous molecularly solubilized enzyme-polymer conjugate solution comprising an organic solvent or an ionic liquid to a vessel, wherein the organic solvent or the ionic liquid comprises no greater than 50 percent water by volume as a co-solvent. The molecularly solubilized enzyme-polymer conjugate comprises covalently bonded polymer chains grown from an enzyme-initiator conjugate and the molecularly solubilized enzyme-polymer conjugate has a hydrodynamic diameter that is less than two-fold of a hydrodynamic diameter of the molecularly solubilized enzyme-polymer conjugate measured in water at a non-denaturing pH. The method further comprises providing at least one reactant to the vessel, and conducting a reaction catalyzed by the enzyme-polymer conjugate.

In various aspects, the non-aqueous molecularly solubilized enzyme-polymer conjugate solution may include an enzyme-polymer conjugate molecularly dissolved in organic solvent or the ionic liquid. For example, the organic solvent or ionic liquid may include an anhydrous organic solvent, or a solvent forming a non-aqueous solution, comprising, for example, acetonitrile, chloroform, tetrahydrofuran, 1,4-dioxane, an ether, hexane, toluene, or acetone, or combinations of any thereof. In various aspects, the solvent may comprise a polar or hydrophilic solvent.

In various aspects, the molecularly solubilized enzyme-polymer conjugate may comprise an enzyme-polymer conjugate synthesized using the grafting-from approach. For example, the molecularly solubilized enzyme-polymer conjugate may comprise an esterase-polymer conjugate comprising a CT-pDMAEMA conjugate, a thermolysin-pOEGMA conjugate, a metalloproteinase-pOEGMA conjugate, a subtilase-ionic liquid polymer conjugate, a subtilisin-ionic liquid polymer conjugate, or a lipase-pDMAA conjugate, or combinations of any thereof.

In various aspects, the reaction catalyzed by the non-aqueous molecularly solubilized enzyme-polymer conjugate solution may comprise, for example, a transesterification reaction, a hydrolysis reaction, an enantioselective reaction, a redox reaction, a condensation reaction, a polyester synthesis reaction, or a peptide synthesis reaction, or combinations of any thereof.

In various aspects, the reaction catalyzed by the non-aqueous molecularly solubilized enzyme-polymer conjugate solution exhibits a k_{cat} of at least 3.7 min⁻¹. In some aspects the reaction catalyzed by the non-aqueous molecularly solubilized enzyme-polymer conjugate solution exhibits a k_{cat} of at least 4 min⁻¹, of at least 5 min⁻¹, of at least 6 min⁻¹, of at least 7 min⁻¹, of at least 8 min⁻¹, of at least 9 min⁻¹, of at least 10 min⁻¹, of at least 15 min⁻¹, of at least 16 min⁻¹, of at least 18 min⁻¹, of at least 20 min⁻¹, of at least 21 min⁻¹, or of at least 25 min⁻¹.

In various aspects, the reaction catalyzed by the non-aqueous molecularly solubilized enzyme-polymer conjugate solution exhibits a K_{M} value less than about 22 mM. In some aspects the reaction catalyzed by the non-aqueous molecularly solubilized enzyme-polymer conjugate solution exhibits a K_{M} value less than about 20 mM, less than about 17 mM, less than about 15 mM, less than about 12 mM, or less than about 10 mM.

### Examples

### Materials

Materials used in an aspect of the invention include α-chymotrypsin (CT) from bovine pancreas (type II), 2-bromo-2-methylpropionyl bromide, β-alanine, N-hydroxysuccinimide, N,N′-diisopropylcarbodiimide, copper(I) bromide, 1,1,4,7,10,10-hexamethyltriethylenetetramine (HMTETA), 2-(dimethylamino)ethyl methacrylate (DMAEMA, passed over a column of basic alumina prior to use), N-succinyl-L-Ala-L-Ala-L-Pro-L-Phe-p-nitroanilide (Suc-AAPF-pNA), bicinchoninic acid (BCA) solution, copper(II) sulfate solution, dichloromethane, ethyl acetate, 2-propanol, diethyl ether, and n-hexane were purchased from Sigma Aldrich and used without further purification. Dialysis tubes (molecular weight cut off, 25, 15, and 1 kDa (Spectra/Por, Spectrum Laboratories Inc.)) were purchased from Fisher Scientific. N-acetyl-L-phenylalanine (AP), thiophenol, isobutyl chloroformate 4,4′-dithiodipyridine (DTDP), triethylamine, dimethyl sulfoxide (DMSO) and 1-propanol were purchased from Sigma Aldrich. Acetonitrile, dichloromethane and ethanol were purchased from Fisher Scientific. Acetonitrile and 1-propanol were dried by distillation under calcium hydride and stored over activated molecular sieves (3 Å, Sigma Aldrich). DMSO was dried by vacuum distillation under calcium hydride and stored over activated molecular sieves.

### Methods

### Procedure for Synthesis of NHS-Functionalized ATRP Initiator

N-2-Bromo-2-methylpropanoyl-β-alanine N′-oxysuccinimide ester was synthesized as follows. Mixture of 2-bromo-2-methylpropionyl bromide (12.4 mL, 100 mmol) and dichloromethane (50 mL) was slowly added into the solution of β-alanine (8.9 g, 100 mmol) and sodium hydrogen carbonate (21 g, 250 mmol) in deionized water (200 mL) at 0° C. then the mixture was stirred at room temperature for 2 h. The water phase was washed with dichloromethane (100 mL×3) and adjusted to pH 2 with 1.0 N HCl aq. at 0° C. The product was extracted with ethyl acetate (150 mL×6). The organic phase was dried with MgSO4 and evaporated to remove solvent. N-2-Bromo-methylpropionyl-β-alanine was isolated by recrystallization from mixture of diethyl ether and n-hexane (1/9 volume ratio). N,N′-diisopropylcarbodiimide (2.8 g, 22 mmol) was slowly added to the solution of N-2-bromo-2-methylpropionyl-β-alanine (4.8 g, 20 mmol), and N-hydroxysuccinimide (2.5 g, 22 mmol) in dichloromethane (200 mL) at 0° C. The mixture was stirred at room temperature for 4 h. After filtering out the precipitated urea, the solution was evaporated to remove solvents. N-2-Bromo-2-methylpropionyl-β-alanine N′-oxysuccinimide ester (1) was purified by recrystallization from 2-propanol. The chemical structure was confirmed by ¹H NMR and IR.

¹H NMR spectra were recorded on a spectrometer (300 MHz, Bruker Avance) with deuterium oxide (D₂O), DMSO-d₆ and CDCl3. Routine FT-IR spectra were obtained with a Nicolet Avatar 360 FT-IR spectrometer (Thermo). UV-vis spectra were obtained and used for enzyme activity determination using an UV-vis spectrometer (Lambda 2, PerkinElmer) with a temperature-controlled cell holder. Melting points (mp) were measured with a Laboratory Devices Mel-Temp. Number and weight average molecular weights (Mₙ and M_{w}) and the polydispersity index (M_{w}/Mₙ) were estimated by gel permeation chromatography (GPC) on a Water 2695 Series with a data processor, equipped with three columns (Waters Ultrahydrogel Linier, 500 and 250), using 100 mM sodium phosphate buffer with 0.2 vol. % trifluoroacetic acid (pH 2.5) as an eluent at a flow rate 0.5 mL/min, with detection by a refractive index (RI) detector. Polyethylene glycol standards were used for calibration.

### Matrix-Assisted Laser Desorption Ionization Time-of-Flight

The molecular weight of the ATRP CT-initiator conjugate was estimated with a Perseptive Biosystems Voyager Elite MALDI-TOF spectrometer.

### Dynamic Light Scattering (DLS)

The DLS data were collected on a Malvern Zetasizer nano-ZS. The concentration of the sample solution was kept at 0.24 mg/mL. The hydrodynamic diameter of the native CT was measured in each organic solvent (i.e., acetonitrile, dichloromethane, chloroform, tetrahydrofuran, and acetone), see Table 1. The hydrodynamic diameter of the CT-pDMAEMA conjugate was measured five times (5 runs/measurement) in each organic solvent, (i.e., acetonitrile, dichloromethane, chloroform, tetrahydrofuran, and acetone), see Table 1.

### Preparation of the Esterase-Initiator Conjugate

CT (1.0 g, 0.56 mmol of amine groups contained) was dissolved in 100 mM sodium phosphate buffer (pH 8.0) at 0° C. After adding the NHS-functionalized TRP initiator (619 mg, 1.85 mmol), the mixture was stirred in a refrigerator for 3 h and the CT-initiator conjugate was isolated by dialysis using a 15 kDa molecular weight cutoff dialysis tube in deionized water in a refrigerator for 24 h and then lyophilized.

### Preparation of Esterase-pDMAEMA Conjugates

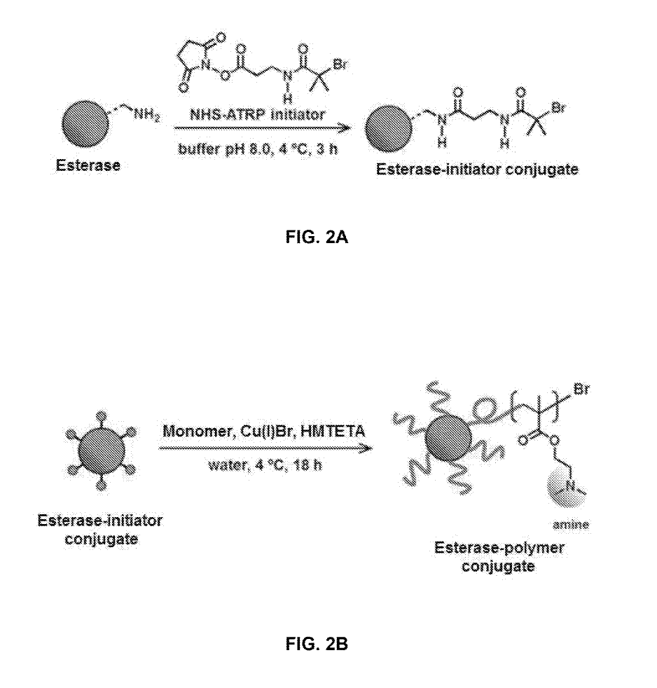

FIGS. 2A and 2B show schematics of the synthesis of an esterase-polymer conjugate, synthesized using the polymer-based protein engineering (PBPE) polymerization method, ATRP and a “grafting-from” approach. An esterase surface-initiated “grafting-from” technique was employed where an initiator was first covalently bound to the esterase (see FIG. 2A) followed by controlled radical polymer synthesis using the esterase-initiator conjugate (see FIG. 2B).

A solution of DMAEMA (169 μL, 1.0 mmol for sample conjugate 1 (C1); 337 μL, 2.0 mmol for C2; 675 μL, 4.0 mmol for C3; 1.35 mL, 8.0 mmol for C4) and CT-initiator conjugate (100 mg, 0.046 mmol of initiator groups) in deionized water (30 mL) was sealed and bubbled with argon in an ice bath for 50 min. Deoxygenated catalyst solutions of HMTETA (55 μL, 0.2 mmol) and Cu(I)Br (29 mg, 0.2 mmol) in deionized water (10 mL) was then added to the conjugation reactor under Argon bubbling. The mixture was sealed and stirred for 18 h at 4° C. to avoid self-polymerization of the DMAEMA. CT-pDMAEMA conjugates were isolated by dialysis with a 25 kDa molecular weight cutoff dialysis tube in deionized water in a refrigerator for 24 h and then lyophilized.

Cleavage of the Grafted pDMAEMA from the Conjugate

CT-pDMAEMA conjugate (10-20 mg) and 6 N HCl aq. (2 to 3 mL) were placed in a hydrolysis tube. After three freeze-pump-thaw cycles, the hydrolysis was performed at 110° C. for 24 h in vacuum. The cleaved polymer was isolated by dialysis using a 1 kDa molecular weight cut off dialysis tube in deionized water and was then lyophilized. The molecular weight of the cleaved polymer was measured by GPC.

### Determination of Molecular Weight of the Prepared Conjugates

Molecular weights of the prepared CT-pDMAEMA conjugates were calculated from estimated molecular weight of the cleaved pDMAEMA from the conjugate. Bicinchoninic Acid Protein Assay (BCA) and absorption assays were also carried out to determine molecular weight of the conjugates.

Using an initiator and ATRP under aqueous conditions, as described above, dense CT-pDMAEMA conjugates were synthesized with relatively narrow molecular weight distributions. The CT-pDMAEMA conjugates had higher relative enzyme activities compared to native CT below pH 8. Indeed, the conjugates had a ten-fold higher enzyme activity than native enzyme at pH 5. Nearly saturated conjugation of the CT enzyme with 12 of 13 potential sites modified was achieved. These results demonstrate that high density polymer conjugation is achievable and that conjugates using responsive polymers can influence enzyme behavior.

### Solubility of the Esterase-Polymer Conjugate, CT-pDMAEMA

In order to achieve the most stable enzyme-polymer conjugate with the largest potential to increase solvation in organic solvents, we examined the solubility and activity in acetonitrile of the largest CT-pDMAEMA conjugate that was prepared. This conjugate consisted of a CT core surrounded by 12 pDMAEMA polymer chains each with an average molar mass of 23.1 kDa. The hydrodynamic diameter (Dₕ) of the conjugate in aqueous media was approximately 34 nm and the overall molar mass of the bioconjugate was 305 kDa.

Prior to examining activity of the CT-pDMAEMA conjugates in anhydrous environments, the effect of polymer conjugation on solubility of the conjugates in acetonitrile and as well as in dichloromethane, chloroform, tetrahydrofuran, and acetone was determined.

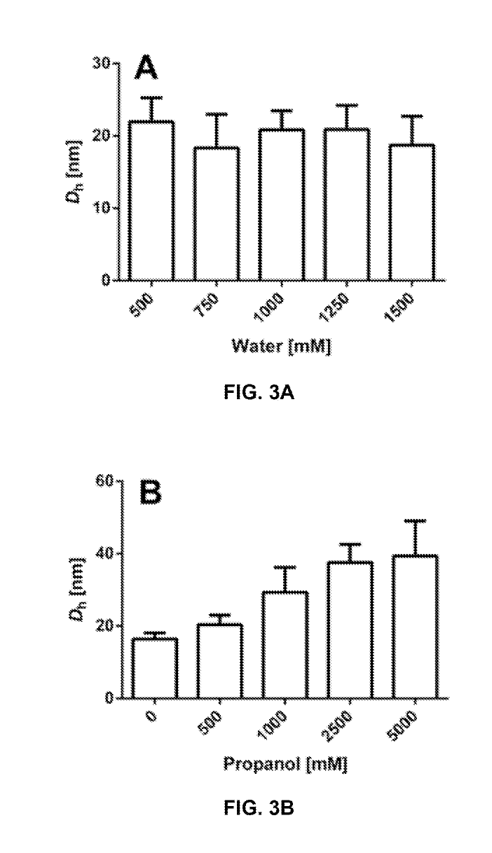

The hydrodynamic diameter values, Dₕ, of the synthesized CT-pDMAEMA conjugates were measured using dynamic light scattering (DLS) in acetonitrile/water/propanol mixtures. FIGS. 3A and 3B show graphs of the measured hydrodynamic diameter values, Dₕ, of the synthesized CT-pDMAEMA conjugates in acetonitrile/water and acetonitrile/propanol respectively. FIG. 3A shows the dependence of the hydrodynamic diameter (Dₕ) of the CT-pDMAEMA on the water content with a fixed propanol concentration (500 mM). FIG. 3B shows the dependence of the hydrodynamic diameter (Dₕ) of the CT-pDMAEMA on the propanol content with a fixed water concentration (1000 mM). The CT-pDMAEMA conjugate was dissolved in acetonitrile at a concentration of 0.24 mg/mL and Dₕ was determined using dynamic light scattering at 25° C. For each of the organic media conditions, CT-pDMAEMA Dₕ was equivalent to that in aqueous conditions, indicating that CT-pDMAEMA was readily dissolved at the molecular scale in essentially anhydrous acetonitrile.

As shown in Table 1, a hydrodynamic diameter study of native CT under the same conditions described above for the enzyme-polymer conjugate, CT-pDMAEMA, formed aggregates as expected, since the native enzyme is insoluble in acetonitrile. CT-pDMAEMA conjugates were also found to be molecularly soluble in dichloromethane, and more soluble than native CT in chloroform, tetrahydrofuran, or acetone (see Table 1). Interestingly, when the propanol concentration was increased with constant water content (1000 mM), the size of CT-pDMAEMA conjugates increased, indicating more extended polymer chains.

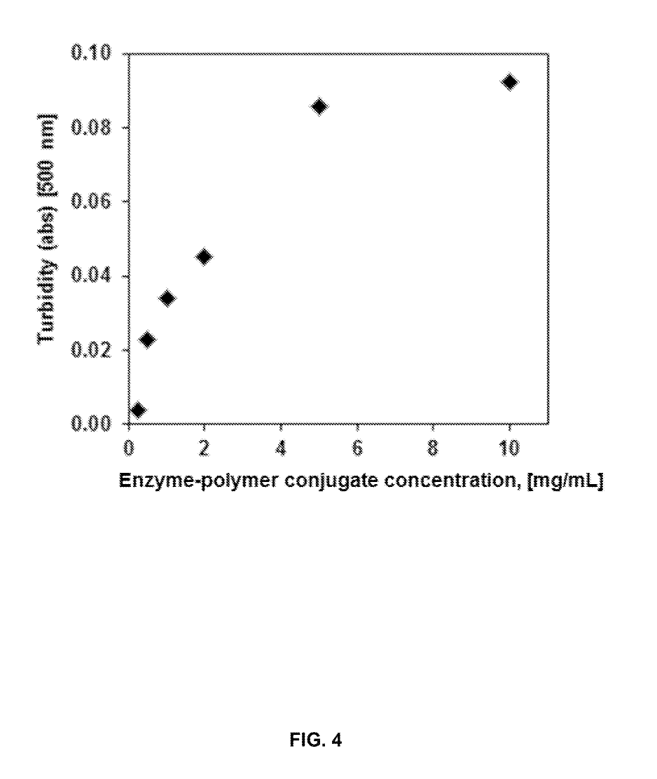

FIG. 4 shows the effect of enzyme-polymer conjugate concentration on turbidity (abs. at 500 nm) in acetonitrile. As shown in FIG. 4 and in Table 1, at each concentration of CT-pDMAEMA conjugate turbidity values were lower than the turbidity value observed for native CT at 0.02 mg/mL. The turbidity measurements observed for the CT-pDMAEMA conjugate indicate that covalent conjugation of pDMAEMA to CT greatly increased the solubility of the CT in acetonitrile. For example, as shown in FIG. 4, even at concentrations approximately 10 times higher than native CT, CT-pDMAEMA conjugates still did not form aggregates in acetonitrile.

TABLE 1
Solubility of CT and CT-pDMAEMA in organic media

| | | Turbidity | Dₕ |
|---|---|---|---|
| Sample | Solvent | (Abs at 500 nm) | (nm) |
| Native CT | Acetonitrile | 0.122 | 510 ± 260 |
| | Dichloromethane | 0.162 | 636 ± 230 |
| | Chloroform | 0.175 | 942 ± 487 |
| | Tetrahydrofuran | 0.218 | 1226 ± 550 |
| | Acetone | 0.205 | 766 ± 236 |
| CT-pDMAEMA | Acetonitrile | 0.004 | 29 ± 7 |
| | Dichloromethane | 0.011 | 21.1 ± 2.0 |
| | Chloroform | 0.050 | 211.7 ± 60.3 |
| | Tetrahydrofuran | 0.184 | 270.0 ± 131.7 |
| | Acetone | 0.332 | 546.6 ± 204.0 |

Comparative solubility of native CT (0.02 mg/mL) and CT-pDMAEMA (0.24 mg conjugate/mL, 0.02 mg enzyme/mL) was determined by measuring turbidity and the number average hydrodynamic diameter (Dₕ) of CT participate suspensions in organic media.

### Kinetics of the CT-pDMAEMA Conjugate in Catalysis

The kinetics of CT polymer conjugate-catalyzed transesterification and hydrolysis of APTE was examined in anhydrous acetonitrile. In order to examine the behavior of ATRP “grown-from” CT-pDMAEMA conjugates in organic media, a thioester substrate for CT was synthesized, APTE.

### Syntheses of APTE and N-Acetyl L-Phenylalanine Propyl Ester (APPE)

The transesterification substrate, APTE, was synthesized from N-acetyl-L-phenylalanine and thiophenol. Isobutyl chloroformate (1.3 mL, 9.7 mmol) was slowly added into a solution of N-acetyl-L-phenylalanine (2.0 g, 9.7 mmol), and triethylamine (1.5 mL, 9.8 mmol) in dichloromethane (100 mL) at 0° C., and the reaction solution was stirred at room temperature for 30 min. A mixture of thiophenol (1.0 mL, 9.6 mmol) in dichloromethane (10 mL) was added to the reaction mixture. After stirring for 30 min, the mixture was washed with 0.1 N HCl aq. (50 mL×2), saturated NaHCO₃ aq. (50 mL×2) and saturated NaCl aq. (50 mL×2). The organic phase was dried over anhydrous MgSO₄ and evaporated to remove dichloromethane. APTE was isolated by recrystallization by ethanol and water (2:1 volume ratio); yield 2.5 g (86%), mp 136-138° C. (lit. 134-136° C.). ¹H NMR (300 MHz, CDCl3) δ 2.02 (s, 3H, acetyl), 3.19 (d, 2H, J=6.6 Hz, CH₂^{β}), 5.13 (td, 1H, J=6.6 and 8.4 Hz, CH^{α}), 5.92 (broad d, 1H, J=8.4 Hz, amide proton), 7.19-7.45 (m, 10H, phenyl) ppm.

APPE was synthesized from N-acetyl-L-phenylalanine and 1-propanol according to the procedure mentioned above. The obtained APPE was isolated by column chromatography on silica gel (70-230 mesh, 60 Å, Sigma Aldrich) using chloroform as eluent. The synthesized APPE was used as a calibration standard for the HPLC studies. ¹H NMR (300 MHz, CDCl3) δ 0.94 (t, 3H, J=7.5 Hz, OCH₂CH₂CH₃), 1.65 (m, 2H, OCH₂CH₂CH₃), 2.01 (s, 3H, acetyl), 3.15 (d, 2H, J=6.9 Hz, CH₂^{β}), 4.09 (t, 2H, J=6.9 Hz, OCH₂CH₂CH₃), 4.90 (m, 1H, H, CH^{α}), 5.93 (broad d, 1H, J=7.8 Hz, amide proton), and 7.11-7.34 (m, 5H, phenyl) ppm.

### Transesterification and Hydrolysis

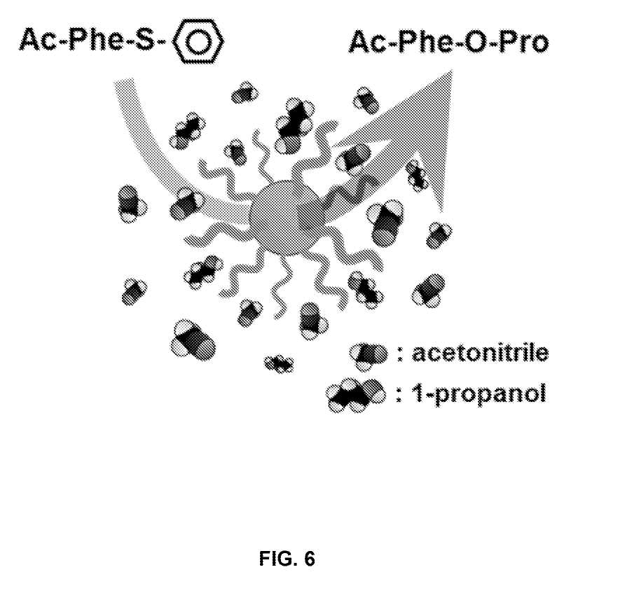

FIG. 5 shows a schematic of the chemical reaction of APPE produced by the CT-pDMAEMA catalyzed transesterification of APTE with 1-propanol in acetonitrile (AN), and the subsequent reaction of thiophenol with DTDP to quantify product formation. FIG. 6 shows a graphic representation of the catalytic activity of the esterase-pDMAEMA conjugate in the chemical reaction of FIG. 5.

Returning back to FIG. 5, the CT-pDMAEMA conjugate also catalyzed the hydrolysis of APTE, resulting in N-acetyl phenylalanine (AP). As a result of both the transesterification and hydrolysis reactions, thiophenol was liberated then subsequently detected and quantified with colorimetric analysis using DTDP.

### Measurement of Transesterification and Hydrolysis Activity by Colorimetric Methods

Colorimetric analysis of transesterification and hydrolysis of APTE by CT-pDMAEMA was carried out in the presence of DTDP reagent. Substrate solution was made by adding APTE (0-500 μL, of 200 mM in a dried acetonitrile, 0-100 mM) to dried acetonitrile (0-500 μL). The substrate solution (500 μL) was then added to 500 μL of CT-pDMAEMA (0.22 mg/mL (0.018 mg of CT/mL) [E]₀=0.7 M), DTDP (0.22 mg/mL, [DTDP]₀=0.98 mM), dried 1-propanol (0-414 μL/mL, [ProOH]₀=0-5000 mM) and water (10-30 μL/mL, [water]=500-1500 mM) solution in dried acetonitrile. The initial rate of transesterification and hydrolysis of APTE at 30° C. was monitored by recording the increasing in absorption at 324 nm (324=11980 M⁻¹ cm⁻¹) using a UV-vis spectrometer.

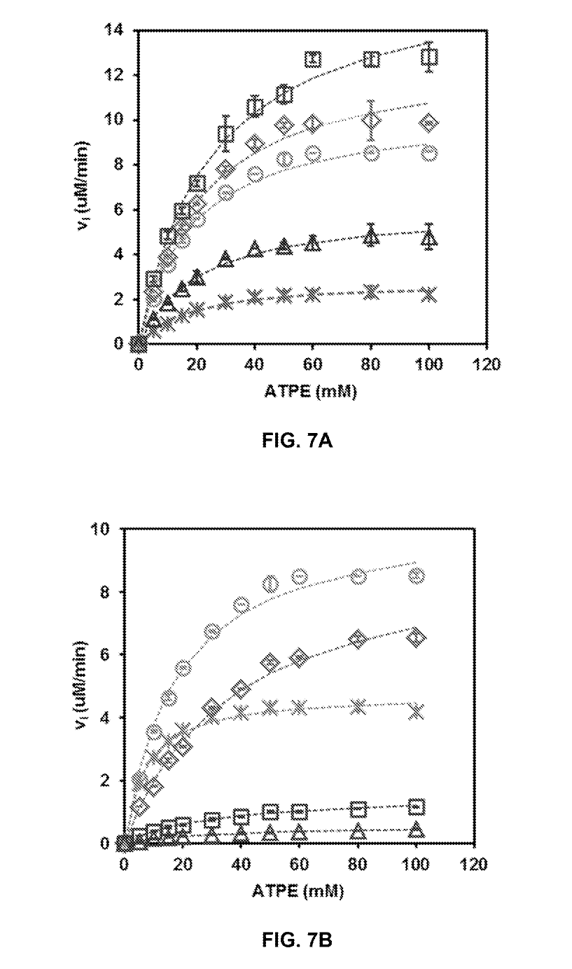

Michaelis-Menten parameters (Vₘₐₓ, k_{cat}, K_{M}) were determined by nonlinear curves versus substrate concentration using the Enzfitter software (as shown in FIGS. 7A and 7B). In more detail, FIGS. 7A and 7B provide the Michaelis-Menten parameters (Vₘₐₓ, k_{cat}, K_{M}) as calculated by Michaelis-Menten curve fitting of APTE against initial velocity plots. The curves shown in FIG. 7A demonstrate the dependence the Michaelis-Menten parameters on the water concentration with a fixed propanol concentration of 500 mM. In FIG. 7A, the open square represents 1500 mM water, the open diamond represents 1250 mM water, the open circle represents 1000 mM water, the open triangle represents 750 mM water, and the asterisk represents 500 mM water. The curves shown in FIG. 7B demonstrate the dependence the Michaelis-Menten parameters on the propanol concentration with a fixed water concentration of 1000 mM. In FIG. 7B, the open triangle represents 5000 mM propanol, the open square represents 2500 mM propanol, the open diamond represents 1000 mM propanol, the open circle represents 500 mM propanol, and the asterisk represents 0 mM propanol.

Specifically, the effect of both water and propanol concentrations on the rate of reaction (k_{cat}) and substrate affinity (Kₘ) was investigated. For a native enzyme suspended in an organic solvent, k_{cat} typically falls by over 3 orders of magnitude and K_{M} increases by at least three orders of magnitude as compared to the results of rate of reaction (k_{cat}) and substrate affinity (K_{M}) observed for the CT-pDMAEMA catalyzed reaction. Since thiophenol is a byproduct of both the transesterification and hydrolysis reactions, DTDP was used to quantify product formation. Thus, the resulting apparent k_{cat} and K_{M} values observed corresponded to the total rate of substrate consumption during the reaction.

The inventors noted unexpected results and were very surprised to observe water-like K_{M} values for the acetonitrile soluble CT-pDMAEMA conjugate. The results were unexpected and surprising because K_{M} values for native enzymes in organic solvents have not been determined accurately because saturation has not been observed. The dramatically low K_{M} values for APTE affinity to the acetonitrile-soluble CT-pDMAEMA conjugate were also insensitive to increasing water concentration. Thus, the total turnover number (k_{cat}) for CT-pDMAEMA catalyzed degradation of APTE increased with increasing water content (see Table 2).

TABLE 2
The effect of water and propanol concentration on apparent
Michaelis-Menten parameters for CT-pDMAEMA catalyzed
transesterification and hydrolysis of APTE.

| Water | Propanol | k_{cat} | K_{M} | k_{cat}/K_{M} |
|---|---|---|---|---|
| (mM) | (mM) | (min⁻¹) | (mM) | (mM⁻¹min⁻¹) |
| 500 | 500 | 3.7 ± 0.1 | 17 ± 1 | 0.21 ± 0.03 |
| 750 | 500 | 8.6 ± 0.3 | 20 ± 2 | 0.43 ± 0.04 |
| 1000 | 500 | 16 ± 0.5 | 22 ± 2 | 0.74 ± 0.07 |
| 1250 | 500 | 18 ± 0.5 | 20 ± 2 | 0.86 ± 0.08 |
| 1500 | 500 | 21 ± 0.6 | 22 ± 2 | 0.95 ± 0.1 |
| 1000 | 500 | 16 ± 0.5 | 22 ± 2 | 0.74 ± 0.07 |
| 1000 | 1000 | 13 ± 0.6 | 36 ± 4 | 0.36 ± 0.04 |
| 1000 | 2500 | 2.3 ± 0.1 | 31 ± 4 | 0.07 ± 0.01 |
| 1000 | 5000 | 0.8 ± 0.03 | 32 ± 2 | 0.03 ± 0.002 |

Table 2 Michaelis-Menten parameters were calculated using DTDP to quantify product formation over time at different propanol concentrations in acetonitrile at 30° C. Substrate (APTE) concentration was varied from 0-100 mM. DTDP reacts with both the hydrolysis and transesterification products; thus, these values describe the combined rate.

### Native CT Activity

Native CT activity was measured in dried acetonitrile with 1000 mM propanol and 1000 mM water. Substrate (APTE) concentration used was 20 mM. Initial rate was measured using a calorimetric method for 5 minutes at 30° C. At enzyme concentrations equivalent to CT-pDMAEMA, native CT had no detectable activity.

As shown in Table 3, native CT displayed no transesterification activity at equivalent enzyme concentrations even when increasing water content.

TABLE 3
Determination of comparative CT activity in acetonitrile

| | Conc. | Initial rate | Initial Rate |
|---|---|---|---|
| sample | (mg/mL) | (μM/min) | (μM/min/mg CT) |
| No enzyme | — | 0.022 | N/A |
| Native CT | 0.20 | 0.27 ± 0.03 | 1.3 ± 0.1 |
| | 0.02 | ND¹ | ND |
| CT-pDMAEMA | 0.24 (0.02 CT) | 3.4 ± 0.4 | 170 ± 20 |
| pDMAEMA | 0.22 | 0.27 ± 0.01 | N/A |

¹No product formation was detected.

When increasing native CT concentration 10-fold (from 0.02 mg/mL to 0.2 mg/mL), the activity of native CT (1.3+0.1 μM/min·mg CT) was still two orders of magnitude below CT-pDMAEMA activity (170±20 μM/min·mg CT). As water content increased, the increase in CT-pDMAEMA activity was likely due to more structural flexibility. Previously, when insoluble CT transesterification and hydrolysis activities were measured in ionic solution with increasing water concentrations in organic solvents, the enzyme exhibited a bell shaped activity curve. Unexpectedly, the inventors found that as the water concentration increased the enzyme-polymer conjugate simply increased activity in acetonitrile (Table 2). It is likely that the dense pDMAEMA shell, generated using “grafting-from” ATRP to surround the enzyme, protected the CT from denaturation at the sufficiently high water concentrations. Both CT-pDMAEMA activity and APTE substrate affinity decreased with increasing propanol concentration (see Table 2). Enzyme activity plausibly decreased as a result of the direct interaction of propanol with the polymer chains attached to surface of the enzyme. The observation of the impact of propanol on the size of these protein-polymer conjugates dissolved in acetonitrile support this finding.

### Measurement of Transesterification and Hydrolysis Activity Using RP-HPLC

The effect of water and propanol concentration on the individual rates of CT-pDMAEMA catalyzed transesterification and hydrolysis was determined using reverse phase-high pressure liquid chromatography (RP-HPLC).

APTE (60 mg, 20 mM) was added to a solution of CT-pDMAEMA (2.2 mg, 0.7 μM of CT) with different amounts of dried 1-propanol (0-5000 mM) and water (500-1500 mM) in dried acetonitrile (10 mL) in screw-capped glass vial, and incubated at 30° C. Aliquots were removed at 1 to 2 h intervals and initial rates were determined from the linear progress by comparison with calibration of AP and APPE.

The initial rates of APPE (transesterification reaction) and AP (hydrolysis reaction) product formation were separately quantified. The initial rates of the reactions were quantified using 500-1500 mM water and 500-5000 mM propanol with a constant substrate (APTE) concentration of 20 mM (see FIGS. 7A and 7B).

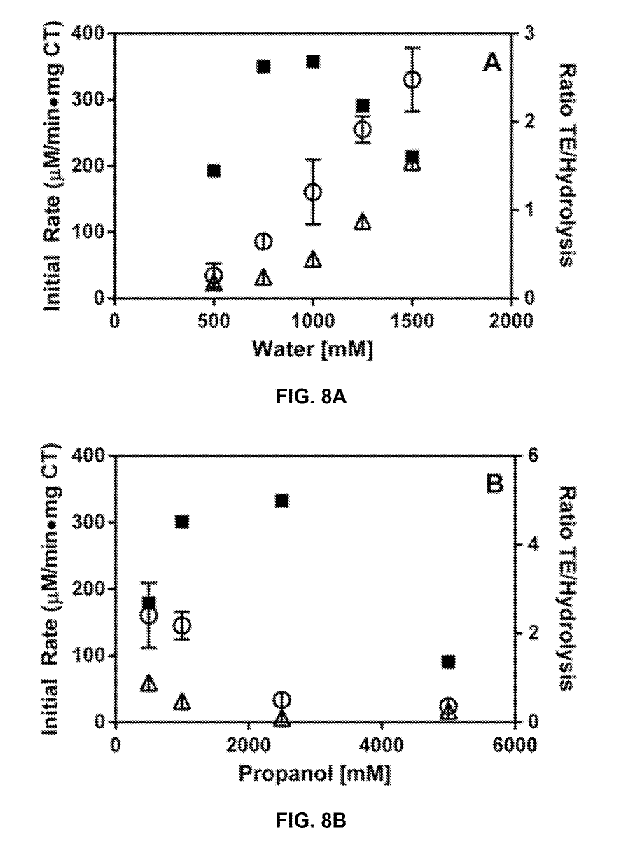

FIG. 8A shows the dependence of CT-pDMAEMA catalyzed transesterification and hydrolysis initial rates on water content with a fixed propanol concentration of 500 mM. FIG. 8B similarly shows the dependence of CT-pDMAEMA catalyzed transesterification and hydrolysis initial rates on propanol content with a fixed water concentration of 1000 mM. In both FIGS. 8A and 8B transesterification rate is represented by an open circle, hydrolysis rate is represented as an open triangle, and the ratio of the transesterification rate/hydrolysis rate is represented as a closed square. The rates of CT-pDMAEMA (0.7 μM) catalyzed transesterification and hydrolysis in acetonitrile at 30° C. were calculated by measuring product formation over time using RP-HPLC.

As shown in FIG. 8A, as water content increased, the initial rates of both transesterification and hydrolysis increased. Conversely, and as shown in FIG. 8B, as propanol concentration was increased, the activity of both reactions decreased. Since it was possible to separately quantify product formation from each reaction, the ratio of transesterification to hydrolysis was calculated. At each concentration, the rate of formation of transesterification products was higher than that of hydrolysis. However, at water concentrations above 1000 mM, CT-pDMAEMA catalyzed hydrolysis rates increased more quickly than CT-pDMAEMA catalyzed transesterification. Thus, when varying water content, we concluded the optimum water concentration was 1000 mM. Finally, we examined the effect of propanol concentration on enzyme activity. Using the optimal water content (1000 mM), we found that a propanol concentration of 2500 mM resulted in the highest transesterification to hydrolysis ratio, although the highest activity for transesterification was at 1000 mM propanol.

In conclusion, we synthesized an enzyme-polymer conjugate (e.g., CT-pDMAEMA conjugate) that showed dramatically increased activity and molecular dissolution in acetonitrile. While other strategies have yielded enzyme activity, those approaches did not enjoy the benefits of molecular dissolution of the enzyme in the organic solvents. Rates for enzyme-polymer conjugate catalyzed transesterification and hydrolysis were proportional to water concentration, and inversely proportional to propanol concentration. Further, the enzyme-polymer conjugates exhibited good substrate binding with APTE (K_{M} as low as 17 mM), and had a specific activity (peak activity 330 μM/min/mg enzyme) many orders of magnitude higher than that of the insoluble native enzyme. Native CT had no detectable activity at equivalent enzyme and substrate concentrations in acetonitrile. The CT-pDMAEMA conjugate had a water-like K_{M} that resembled that of the native enzyme. The molecular dissolution of an active enzyme in an organic solvent used in organic syntheses represents a significant step toward application of non-aqueous enzymology.

It should be understood that this disclosure is not limited to the various aspects or embodiments disclosed herein, and it is intended to cover modifications that are within the spirit and scope of the invention, as defined by the claims.

We claim:

1. A non-aqueous solution comprising:
an organic solvent or an ionic liquid, wherein the organic solvent or the ionic liquid comprises no greater than 50 percent water by volume as a co-solvent; and
an enzyme-polymer conjugate molecularly dissolved in the organic solvent or the ionic liquid,
wherein the enzyme-polymer conjugate comprises covalently bonded polymer chains grown from an enzyme-initiator conjugate, and
wherein the molecularly dissolved enzyme-polymer conjugate in the non-aqueous solution has a hydrodynamic diameter that is less than two-fold of a hydrodynamic diameter of the enzyme-polymer conjugate molecularly dissolved in water at a non-denaturing pH.

2. The non-aqueous solution of claim 1, wherein the enzyme-polymer conjugate comprises an esterase-polymer conjugate.

3. The non-aqueous solution of claim 1, wherein the organic solvent or the ionic liquid comprises greater than 50 percent by volume acetonitrile, chloroform, tetrahydrofuran, 1,4-dioxane, an ether, hexane, toluene, or acetone, or combinations of any thereof.

4. The non-aqueous solution of claim 1, wherein the organic solvent comprises an anhydrous organic solvent.

5. The non-aqueous solution of claim 1, wherein the enzyme-polymer conjugate comprises an esterase-polymer conjugate comprising a CT-pDMAEMA conjugate, a metalloproteinase-pOEGMA conjugate, a subtilase-ionic liquid polymer conjugate, or a lipase-pDMAA conjugate, or combinations of any thereof.

6. The non-aqueous solution of claim 1, wherein the molecularly dissolved enzyme-polymer conjugate comprises an enzyme capable of catalyzing a transesterification reaction, a hydrolysis reaction, an enantioselective reaction, a redox reaction, a condensation reaction, a polyester synthesis reaction, or a peptide synthesis reaction, or combinations of any thereof.

7. A method of catalyzing a reaction comprising:
dissolving an enzyme-polymer conjugate in an organic solvent or an ionic liquid to form a molecular solution of the enzyme-polymer conjugate in the organic solvent or the ionic liquid
wherein the enzyme-polymer conjugate comprises covalently bonded polymer chains grown from an enzyme-initiator conjugate, and
wherein the dissolved enzyme-polymer conjugate in the molecular solution has a hydrodynamic diameter that is less than two-fold of a hydrodynamic diameter of the enzyme-polymer conjugate molecularly dissolved in water at a non-denaturing pH; and
conducting a reaction catalyzed by the enzyme-polymer conjugate in the organic solvent or the ionic liquid.

8. The method of claim 7, wherein the organic solvent or the ionic liquid comprises acetonitrile, chloroform, tetrahydrofuran, 1,4-dioxane, an ether, hexane, toluene, or acetone, or combinations of any thereof.

9. The method of claim 7, wherein the organic solvent comprises an anhydrous organic solvent.

10. The method of claim 7, wherein the enzyme-polymer conjugate comprises an esterase-polymer conjugate comprising a CT-pDMAEMA conjugate, a metalloproteinase-pOEGMA conjugate, a subtilase-ionic liquid polymer conjugate, or a lipase-pDMAA conjugate, or combinations of any thereof.

11. The method of claim 7, wherein the reaction comprises a transesterification reaction, a hydrolysis reaction, an enantioselective reaction, a redox reaction, a condensation reaction, a polyester synthesis reaction, or a peptide synthesis reaction, or combinations of any thereof.

12. A method of catalyzing a reaction comprising:
providing a non-aqueous molecularly solubilized enzyme-polymer conjugate solution comprising an organic solvent or an ionic liquid to a vessel,
wherein the organic solvent or the ionic liquid comprises no greater than 50 percent water by volume as a co-solvent,
wherein the molecularly solubilized enzyme-polymer conjugate comprises covalently bonded polymer chains grown from an enzyme-initiator conjugate, and
wherein the molecularly solubilized enzyme-polymer conjugate in the solution has a hydrodynamic diameter that is less than two-fold of a hydrodynamic diameter of the molecularly solubilized enzyme-polymer conjugate measured in water at a non-denaturing pH;
providing at least one reactant to the vessel; and
conducting a reaction catalyzed by the enzyme-polymer conjugate.

13. The method of claim 12 wherein the organic solvent or the ionic liquid comprises acetonitrile, chloroform, tetrahydrofuran, 1,4-dioxane, an ether, hexane, toluene, or acetone, or combinations of any thereof.

14. The method of claim 12, wherein the organic solvent comprises an anhydrous organic solvent.

15. The method of claim 12, wherein the molecularly solubilized enzyme-polymer conjugate comprises an esterase-polymer conjugate comprising a CT-pDMAEMA conjugate, a metalloproteinase-pOEGMA conjugate, a subtilase-ionic liquid polymer conjugate, or a lipase-pDMAA conjugate, or combinations of any thereof.

16. The method of claim 12, wherein the reaction comprises a transesterification reaction, a hydrolysis reaction, an enantioselective reaction, a redox reaction, a condensation reaction, a polyester synthesis reaction, or a peptide synthesis reaction, or combinations of any thereof.
